(19) United States
(12) Patent Application Publication
(10) Pub. No.: US 2024/0049670 A1
(43) Pub. Date: Feb. 15, 2024

(54) TISSUE CULTURED CANNABIS PLANTLET AND METHODS FOR PREPARING SAME

(71) Applicants: CANNADORF LTD., Tel Aviv (IL); YISSUM RESEARCH DEVELOPMENT COMPANY OF THE HEBREW UNIVERSITY OF JERUSALEM LTD., Jerusalem (IL)

(72) Inventors: Yaacov BUXDORF, Ness-Ziona (IL); Leor ESHED WILLIAMS, Kidron (IL); Marganit LEVY, Rehovot (IL)

(73) Assignees: CANNADORF LTD., Tel Aviv (IL); YISSUM RESEARCH DEVELOPMENT COMPANY OF THE HEBREW UNIVERSITY OF JERUSALEM LTD., Jerusalem (IL)

(21) Appl. No.: 18/270,792

(22) PCT Filed: Jan. 3, 2022
(86) PCT No.: PCT/IL2022/050007

§ 371 Date: Jul. 3, 2023

Related U.S. Application Data
(60) Provisional application No. 63/133,357, filed on Jan. 3, 2021.

(51) Int. Cl. A01H 6/28; A01H 4/00; A01H 5/12

(52) U.S. Cl. CPC A01H 6/28; A01H 4/00; A01H 5/12

(57) ABSTRACT

The present invention is directed to a tissue cultured Cannabis plantlet, including methods for producing, and using same.

### CROSS-REFERENCE TO RELATED-APPLICATIONS

This application claims the benefit of priority of U.S. Provisional Application No. 63/133,357, titled “METHODS FOR CONTROLLING CANNABINOIDS PRODUCTION”, filed Jan. 3, 2021, the contents of which are incorporated herein by reference in their entirety.

### FIELD OF INVENTION

The present invention relates to methods for controlled production of cannabinoid compounds, in tissue cultures, e.g., semi tissue culture.

### BACKGROUND

Cannabis sativa (Cannabis) was originally discovered in central Asia, and it is one of humanity's oldest crops. Thanks to its secondary metabolites content, Cannabis has been used in Ayurvedic Medicine for more than 3,000 years, Chinese Medicine for more than 2,000 years and used in Allopathic (Western) Medicine since the beginning of the 19^{th} century. Later during the 20^{th} century, the use of Cannabis became illegal in many countries due to its psychoactive effects. Today Cannabis prohibition is being lifted across the globe and at least 24 nations have legalized Cannabis: for medicine, recreation, or for both. In the United States, 46 States have legalized Cannabis in some form with the projection of near future legalization across all the United States of America.

The Cannabis plant produces numerous secondary metabolites including cannabinoids and terpenes, which are known for their therapeutic effect. Many of these metabolites are produced in a special structure, termed glandular trichome, developed mostly on leaves called bract that encapsulates the female's reproductive parts in the female flower. The cola, which is a cluster of flower buds (inflorescences) that grow tightly together, develops upon induction of flowering on every growing tip, emerging from leaf nodes along the stem. As the flower develops the trichrome go through ripening process having three visible stages: translucent, opaque, and amber, simultaneously with a change in the content of the secondary metabolites.

Cannabinoids are extracted from inflorescences located at different positions on the plant, bearing trichomes at various developmental stages, each displaying a distinct composition of secondary metabolites, which altogether introduces great variability to the cannabinoids profile. Subsequently, the lack of standardization leads to inconsistency in the therapeutic effect.

The attempt to treat patients with synthetic cannabinoid yielded no results and it is well established that extracts from raw Cannabis are more potent than synthetic cannabinoids, due to the various active molecules that act synergistically.

Currently, it is impossible to link chemical profile to disease alleviation due to the inconsistency in plant extracts.

Therefore, there is still a great need for increased reproducibility in processes for producing cannabinoids, terpenes, flavonoids, or a combination thereof.

### SUMMARY

According to a first aspect, there is provided a tissue cultured Cannabis plantlet comprising an inflorescence (IF), and characterized by any one of: (a) comprising leaves having a cuticle being at least 5% less thick compared to a cuticle of leaves of a control mother plant; (b) being devoid of a root or a rooting system; and (c) a combination of (a) and (b).

According to another aspect, there is provided a plurality of IF derived or obtained from the tissue cultured Cannabis plantlet disclosed herein, characterized by a production profile of at least one cannabinoid, at least one terpene, at least one flavonoid, or any combination thereof, being essentially the same among the plurality of IF.

According to another aspect, there is provided a method for producing a tissue cultured Cannabis plantlet disclosed herein, the method comprising the steps of: (a) providing a nodal explant derived or obtained from a Cannabis mother plant; and (b) culturing the nodal explant under at least partially under semi-immersion conditions, on agar, or both, thereby, producing the tissue cultured Cannabis plantlet.

According to another aspect, there is provided a method for controlling the production profile of at least one cannabinoid, at least one terpene, at least one flavonoid, or any combination thereof, the method comprising providing the tissue cultured Cannabis plantlet disclosed herein, and subjecting the cultured Cannabis plantlet to conditions suitable for modifying the production profile of at least one cannabinoid, at least one terpene, at least one flavonoid, or any combination thereof.

In some embodiments, the leaves of the tissue cultured Cannabis plantlet are characterized by having at least 15% by weight less pigment compared to leaves of the control mother plant.

In some embodiments, the pigment comprises: a chlorophyll a, a chlorophyll b, a carotenoid, or any combination thereof.

In some embodiments, the leaves of the tissue cultured Cannabis plantlet are characterized by having at least 35% by weight less chlorophyll a, compared to leaves of the control mother plant.

In some embodiments, the leaves of the tissue cultured Cannabis plantlet are characterized by having at least 45% by weight less chlorophyll b, compared to leaves of the control mother plant.

In some embodiments, the leaves of the tissue cultured Cannabis plantlet are characterized by having at least 25% by weight less total chlorophyll content, compared to leaves of the control mother plant.

In some embodiments, the leaves of the tissue cultured Cannabis plantlet are characterized by having at least 10% by weight more fatty acids compared to leaves of the control mother plant.

In some embodiments, the amount of the fatty acid is determined in an extract of the leaves of the tissue cultured Cannabis plantlet.

In some embodiments, the leaves comprises: fan leaves, sugar leaves, or both, of the tissue cultured Cannabis plantlet.

In some embodiments, the tissue cultured Cannabis plantlet is devoid of a pathogen or a part derived therefrom, a pesticide, or both.

In some embodiments, the pathogen comprises: a bacterium, a fungus, a virus, a protozoa, a metazoan, or any combination thereof.

In some embodiments, the part comprises: a spore, a mycelium, an egg, a pupa, a larva, a nymph, or any combination thereof, of the pathogen.

In some embodiments, the control mother plant comprises a whole plant grown indoor or in the field.

In some embodiments, the plurality of IF is obtained or derived from a plurality of tissue cultured Cannabis plantlets disclosed herein.

In some embodiments, the culturing at least partially under semi-immersion conditions, is in a semi-immersion bioreactor, on agar substrate, or both.

In some embodiments, the step (b) further comprises contacting the nodal explant with an effective amount of at least one elicitor selected from the group consisting of: bacterial flagella peptide (F1g22), chitin, chitosan, methyl jasmonate (MeJA), and Botrytis cinerea-derived material, and any combination thereof.

In some embodiments, the culturing under semi-immersion conditions, on agar, or both, is at least 7 days.

In some embodiments, the controlling comprises increasing the reproducibility of the produced profile of at least one cannabinoid, at least one terpene, at least one flavonoid, or any combination thereof, compared to a control mother plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1K includes micrographs showing micropropagation of Cannabis. (1A-1C) Pieces of Cannabis stem (one node) were sterilized and entered into tissue culture boxes. On (1C) A plantlet that grew in boxes two weeks later. (1D-1G) Plantlets that were transferred into rooting media in different type of vessels. (1H) nodes after sterilization. (1I-1J) Sixteen (16) nodes were placed in a semi-immersion bioreactor. (1K) Two weeks later the plantlets were taken out.

FIGS. 2A-2D includes images showing that a Cannabis plantlet of the invention (2A-2B) is enriched with trichomes (2C-2D).

FIGS. 3A-3B include images showing a Cannabis plantlet of the invention cultured by means of semi-immersion (3A) or on a solid substrate (3B).

FIGS. 4A-4B include images showing leaves taken from (4A) TA5 plant from tissue a culture and (4B) TA5 plant from growth room. Fan leaves marked with white arrows and sugar leaves marked with white arrowheads.

FIG. 5 includes a vertical bar graph showing the stomata average number in 0.15 mm² abaxial Cannabis sativa fan leaves grown in tissue culture (TC) as compared to growth room (GR). Stomata were counted in light microscope. Shown are averages and standard errors. n=10, asterisk (*) denotes statistical differences according to student's t-test, p<0.01.

FIGS. 6A-6B include micrographs showing stomata in abaxial Cannabis sativa fan leaves (6A) grown in tissue culture (TC) as compared to (6B) growth room (GR). Pictures were taken using scanning electron microscopy (SEM), scale bar=50 μm.

FIG. 7 includes a vertical bar graph showing cuticle thickness in Cannabis sativa sugar leaves grown in tissue culture (TC) as compared to growth room (GR). Tissue was fixed and scanned in SEM. Shown are averages and standard errors. n=11-16, asterisk denotes statistical differences according to student's t-test, p<0.01.

FIGS. 8A-8B include micrographs showing cuticle thickness in Cannabis sativa sugar leaves grown in (8A) tissue culture (TC) as compared to (8B) growth room (GR). Pictures were taken using SEM, bar=10 μm.

FIG. 9 includes a vertical bar graph showing amount of fatty acids in extracts of cuticle of Cannabis sativa sugar leaves grown in tissue culture (TC), compared to growth room (GR).

FIGS. 10A-10C include vertical bar graphs showing amounts of (10A) Chlorophyll a, (10B) chlorophyll b, and (10C) total carotenoids, in extracts of Cannabis sativa sugar leaves grown in tissue culture (TC) as compared to growth room (GR). Shown are averages and standard errors. n=3, asterisk denotes statistical differences by student t-test, p<0.01.

FIG. 11 include images of tissue cultured Cannabis plantlets as disclosed herein. Scale bar=1 cm.

### DETAILED DESCRIPTION

According to some embodiments, there is provided a tissue cultured Cannabis plantlet.

In some embodiments, the tissue cultured Cannabis plantlet comprises leaves being characterized by having a cuticle being at least 5% less thick compared to a cuticle of a leaf of a control mother plant.

In some embodiments, the tissue cultured Cannabis plantlet is characterized by being devoid of a root or a rooting system.

In some embodiments, the tissue cultured Cannabis plantlet comprises leaves being characterized by having a cuticle being at least 5% less thick compared to a cuticle of a leaf of a control mother plant and by being devoid of a root or a rooting system.

In some embodiments, the leaves of the tissue cultured Cannabis plantlet are characterized by having a cuticle being at least 1%, at least 2%, at least 3%, at least 4%, at least 5%, at least 7%, at least 10%, at least 15%, or at least 20% less thick compared to a cuticle of a leaf of a control mother plant, or any value and range therebetween. Each possibility represents a separate embodiment of the invention.

In some embodiments, at least 20%, at least 30%, at least 40%, at least 50%, at least 60%, at least 70%, at least 80%, at least 90%, or at least 99% of the leaves of the tissue cultured Cannabis plantlet are characterized by having a cuticle being at least 1%, at least 2%, at least 3%, at least 4%, at least 5%, at least 7%, at least 10%, at least 15%, or at least 20% less thick compared to a cuticle of a leaf of a control mother plant, or any value and range therebetween. Each possibility represents a separate embodiment of the invention.

In some embodiments, a leaf comprises a sugar leaf. In some embodiments, a leaf comprises a fan leaf. In some embodiments, a leaf comprises a sugar leaf and a fan leaf. In some embodiments, a leaf comprises a plurality of leaves. In some embodiments, a plurality of leaves comprises at least two leaves of the same type, as disclosed herein, such as fan, or sugar. In some embodiments, a plurality of leaves comprises a plurality of types of leaves, such as fan and sugar.

In some embodiments, the tissue cultured Cannabis plantlet comprises leaves comprising at least 5% by weight less, at least 15% by weight less, at least 25% by weight less, at least 50% by weight less, at least 60% by weight less, at least 70% by weight less, at least 80% by weight less, at least 90% by weight less, at least 95% by weight less, or at least 99% by weight less pigment compared to a control leaf of a mother plant, or any value and range therebetween. Each possibility represents a separate embodiment of the invention.

In some embodiments, the tissue cultured Cannabis plantlet comprises leaves comprising at least 15% by weight less pigment compared to a control leaf of a mother plant, or any value and range therebetween. Each possibility represents a separate embodiment of the invention.

In some embodiments, a pigment comprises: a chlorophyll a, a chlorophyll b, a carotenoid, or any combination thereof.

In some embodiments, the leaves of a tissue cultured Cannabis plantlet as disclosed herein are characterized by having or comprising at least 10% by weight less, at least 15% by weight less, at least 20% by weight less, at least 25% by weight less, at least 30% by weight less, at least 35% by weight less, at least 40% by weight less, or 50% by weight less chlorophyll a, compared to leaves of a control mother plant, or any value and range therebetween. Each possibility represents a separate embodiment of the invention. In some embodiments, the leaves of a tissue cultured Cannabis plantlet as disclosed herein are characterized by having or comprising 10% to 50% by weight less chlorophyll a.

In some embodiments, the leaves of a tissue cultured Cannabis plantlet as disclosed herein are characterized by having or comprising at least 35% by weight less chlorophyll a, compared to leaves of a control mother plant.

In some embodiments, the leaves of a tissue cultured Cannabis plantlet as disclosed herein are characterized by having or comprising at least 20% by weight less, at least 25% by weight less, at least 30% by weight less, at least 35% by weight less, at least 40% by weight less, at least 45% by weight less, at least 50% by weight less, or 55% by weight less chlorophyll b, compared to leaves of a control mother plant, or any value and range therebetween. Each possibility represents a separate embodiment of the invention. In some embodiments, the leaves of a tissue cultured Cannabis plantlet as disclosed herein are characterized by having or comprising 20% to 60% by weight less chlorophyll b.

In some embodiments, the leaves of a tissue cultured Cannabis plantlet as disclosed herein are characterized by having or comprising at least 45% by weight less chlorophyll b, compared to leaves of a control mother plant.

In some embodiments, the leaves of a tissue cultured Cannabis plantlet as disclosed herein are characterized by having or comprising at least 10% by weight less, at least 15% by weight less, at least 20% by weight less, at least 25% by weight less, at least 30% by weight less, at least 35% by weight less, at least 40% by weight less, or 50% by weight less total chlorophyll content, compared to leaves of a control mother plant, or any value and range therebetween. Each possibility represents a separate embodiment of the invention. In some embodiments, the leaves of a tissue cultured Cannabis plantlet as disclosed herein are characterized by having or comprising 15% to 40% by weight less total chlorophyll content.

In some embodiments, the leaves of a tissue cultured Cannabis plantlet as disclosed herein are characterized by having or comprising at least 25% by weight less total chlorophyll content, compared to leaves of a control mother plant.

In some embodiments, leaves of a tissue cultured Cannabis plantlet as disclosed herein are characterized by having or comprising at least 4% by weight more, at least 5% by weight more, at least 8% by weight more, at least 10% by weight more, at least 15% by weight more, at least 20% by weight more, at least 25% by weight more, or at least 30% by weight more fatty acids compared to leaves of said control mother plant, or any value and range therebetween. Each possibility represents a separate embodiment of the invention. In some embodiments, the leaves of a tissue cultured Cannabis plantlet as disclosed herein are characterized by having or comprising 5% to 20% by weight more fatty acids compared to leaves of a control mother plant.

In some embodiments, leaves of a tissue cultured Cannabis plantlet as disclosed herein are characterized by having or comprising at least 10% by weight more fatty acids compared to leaves of a control mother plant.

In some embodiments, the amount of fatty acid is determined in an extract obtained or derived from leaves of a tissue cultured Cannabis plantlet, as disclosed herein.

Methods for determining pigment content and/or level, as well of fatty acids are common and would be apparent to one of ordinary skill in the art. Non-limiting example of such method includes, but is not limited to, gas-chromatography mass-spectrometry (GC-MS), spectrophotometry, as exemplified herein.

In some embodiments, the present invention is directed to a pathogen free cannabinoids production. In some embodiments, the tissue cultured Cannabis plantlet disclosed herein, is devoid of pollen. In some embodiments, the tissue cultured Cannabis plantlet disclosed herein, is not fertilized. In some embodiments, the tissue cultured Cannabis plantlet disclosed herein, does not undergo fertilization.

In some embodiments, the tissue cultured Cannabis plantlet of the invention is devoid of a pathogen or a part derived therefrom, a pesticide, or any combination thereof.

In some embodiments, the pathogen comprises: a bacterium, a fungus, a virus, a protozoa, a metazoan, or any combination thereof.

In some embodiments, the pathogen is an arthropod.

In some embodiments, the pathogen is an insect.

In some embodiments, the pathogen part comprises: a spore, a mycelium, an egg, a pupa, a larva, a nymph, any developmental stage thereof, or any combination thereof, of the pathogen.

part comprises: a spore, a mycelium, an egg, a pupa, a larva, a nymph, or any combination thereof, of the pathogen.

In some embodiments, the pathogen is a pest.

As used herein, the terms “pathogen”, “phytopathogen”, and “pest” are interchangeable, and refer to any organism, or any part derived therefrom, that is pathogenic to a plant.

In some embodiments, the tissue cultured Cannabis plantlet of the invention comprises at least one inflorescence (IF). In some embodiments, the tissue cultured Cannabis plantlet of the invention comprises a plurality of IF. In some embodiments, the IF of the tissue cultured Cannabis plantlet of the invention consist essentially of apical IF. In some embodiments, the IF of the tissue cultured Cannabis plantlet of the invention consist of apical IF. In some embodiments, the IF of the tissue cultured Cannabis plantlet of the invention is devoid of lateral IF. In some embodiments, the IF of the tissue cultured Cannabis plantlet of the invention is essentially devoid of lateral IF.

In some embodiments, the tissue cultured Cannabis plantlet comprises a single terminal IF. In some embodiments, the tissue cultured Cannabis plantlet comprises a single apical IF.

According to some embodiments, the tissue cultured Cannabis plantlet of the invention further comprises a stalk or a stem. In some embodiments, the tissue cultured Cannabis plantlet of the invention comprises the IF disclosed herein, and a stalk or a stem.

In some embodiments, the stalk or stem of the tissue cultured Cannabis plantlet disclosed herein, comprises 1 leaf at most, 2 leaves at most, 3 leaves at most, 4 leaves at most, 5 leaves at most, or any value and range therebetween. Each possibility represents a separate embodiment of the invention.

In some embodiments, the stalk or stem of the tissue cultured Cannabis plantlet disclosed herein, comprises 1 to 2 leaves, 1 to 3 leaves, 1 to 4 leaves, 2 to 3 leaves, 2 to 4 leaves, 2 to 5 leaves, 3 to 4 leaves, 3 to 5 leaves, or 4 to 5 leaves. Each possibility represents a separate embodiment of the invention.

In some embodiments, the stalk or stem of the tissue cultured Cannabis plantlet disclosed herein, comprises 1 to 2 leaf lines, 1 to 3 leaf lines, 1 to 4 leaf lines, 2 to 3 leaf lines, 2 to 4 leaf lines, 2 to 5 leaf lines, 3 to 4 leaf lines, 3 to 5 leaf lines, or 4 to 5 leaf lines. Each possibility represents a separate embodiment of the invention.

In some embodiments, the leaves of or on the stalk or stem of the tissue cultured Cannabis plantlet disclosed herein, are located or positioned below or lower than the IF of the tissue cultured Cannabis plantlet.

As used herein, lower or below is on the longitudinal axis of the tissue cultured Cannabis plantlet.

As used herein, the terms “stalk” and “stem” are interchangeable.

In some embodiments, the tissue cultured Cannabis plantlet comprises a stalk or a stem having an average length of at least 0.5 cm, at least 1.0 cm, at least 1.5 cm, at least 2.0 cm, or any value and range therebetween. Each possibility represents a separate embodiment of the invention.

In some embodiments, the tissue cultured Cannabis plantlet comprises the IF disclosed herein having an average length of at least 1.0 cm, at least 1.5 cm, at least 2.0 cm, at least 2.5 cm, at least 3.0 cm, or any value and range therebetween. Each possibility represents a separate embodiment of the invention.

In some embodiments, the tissue cultured Cannabis plantlet disclosed herein has an average length of at least 2 cm, at least 3 cm, at least 4 cm, at least 5 cm, at least 6 cm, at least 7 cm, at least 8 cm, at least 10 cm, at least 12 cm, at least 15 cm, or at least 19 cm, or any value and range therebetween. Each possibility represents a separate embodiment of the invention. In some embodiments, the tissue cultured Cannabis plantlet disclosed herein has an average length of 2 to 10 cm, 3 to 15 cm, 4 to 16 cm, or 5 to 20 cm. Each possibility represents a separate embodiment of the invention.

In some embodiments, the tissue cultured Cannabis plantlet disclosed herein is characterized by having an average ratio of the length of the IF disclosed herein to the total length of the plantlet of at least 0.5, at least 0.55, at least 0.6, at least 0.65, at least 0.7, at least 0.75, at least 0.8, or any value and range therebetween. Each possibility represents a separate embodiment of the invention.

In some embodiments, the tissue cultured Cannabis plantlet comprises a stalk or a stem having an average weight of at least 0.01 gr, at least 0.03 gr, at least 0.05 gr, at least 0.07 gr, at least 0.09 gr, at least 0.10 gr, at least 0.15 gr, at least 0.20 gr, or any value and range therebetween. Each possibility represents a separate embodiment of the invention.

In some embodiments, the tissue cultured Cannabis plantlet comprises the IF disclosed herein having an average weight of at least 0.1 gr, at least 0.25 gr, at least 0.5 gr, at least 1.0 gr, at least 1.25 gr, at least 1.5 gr, or any value and range therebetween. Each possibility represents a separate embodiment of the invention.

In some embodiments, the tissue cultured Cannabis plantlet disclosed herein has an average weight of at least 0.2 gr, at least 0.3 gr, at least 0.4 gr, at least 0.5 gr, at least 0.65 gr, at least 0.75 gr, at least 0.9 gr, at least 1.0 gr, at least 1.1 gr, at least 1.25 gr, at least 1.5 gr, at least 1.6 gr, at least 1.8 gr, or any value and range therebetween. Each possibility represents a separate embodiment of the invention.

In some embodiments, the tissue cultured Cannabis plantlet disclosed herein is characterized by having an average ratio of the weight of the IF disclosed herein to the weight of the plantlet of at least 0.5, at least 0.6, at least 0.7, at least 0.8, at least 0.9, at least 0.95, or any value and range therebetween. Each possibility represents a separate embodiment of the invention.

In some embodiments, the tissue cultured Cannabis plantlet of the invention is characterized an IF to plantlet weight percent of at least 50%, at least 55%, at least 60%, at least 65%, at least 70%, at least 75%, at least 80%, at least 85%, at least 90%, at least 95%, or any value and range therebetween. Each possibility represents a separate embodiment of the invention.

In some embodiments, the IF disclosed herein constitutes at least 50%, at least 55%, at least 60%, at least 65%, at least 70%, at least 75%, at least 80%, at least 85%, at least 90%, or at least 95% by weight of the tissue cultured Cannabis plantlet disclosed herein, or any value and range therebetween. Each possibility represents a separate embodiment of the invention.

In some embodiments, the tissue cultured plantlet of the invention and the mother plant are of the same variety. In some embodiments, the tissue cultured plantlet of the invention and the mother plant are of the same variety and are grown under essentially the same conditions. In some embodiments, the tissue cultured plantlet of the invention and the mother plant are grown under essentially the same conditions. In some embodiments, the tissue cultured plantlet of the invention is obtained or derived from the mother plant. In some embodiments, the tissue cultured Cannabis plantlet of the invention is a plant part obtained or derived from a mother plant, being cultured according to the method disclosed herein, so as to produce or prepare the tissue cultured Cannabis plantlet of the invention.

In some embodiments, a control mother plant comprises a whole plant grown indoor, in the field, or a combination thereof.

According to some embodiments, there is provided a plurality of IF derived or obtained from the tissue cultured Cannabis plantlet as disclosed herein, being characterized by a production profile of at least one cannabinoid, at least one terpene, at least one flavonoid, or any combination thereof, being essentially the same among the plurality of IF.

As used herein, the term “plurality” encompasses any integer equal to or greater than 2. In some embodiments, a plurality comprises at least 2, at least 5, at least 10, at least 20, at least 50, at least 100, at least 200, at least 300, at least 500, at least 700, or at least 1,000, or any value and range therebetween. Each possibility represents a separate embodiment of the invention.

Methods

In some embodiments, the present invention provides methods for producing cannabinoids. In some embodiments, the method comprises producing a predetermined combination of cannabinoids. In some embodiments, the present invention is directed to a method for producing a particular combination of cannabinoids, terpenes, flavonoids, or any combination thereof, upon demand. In some embodiments, the present invention comprises culturing a Cannabis tissue, e.g., a microflowering as described hereinbelow, for the controlled production of cannabinoids, terpenes, flavonoids, or any combination thereof.

According to some embodiments, there is provided a method for producing or preparing a tissue cultured Cannabis plantlet.

In some embodiments, the method comprises the steps of: (a) providing a nodal explant derived or obtained from a Cannabis mother plant; and (b) culturing the nodal explant at least partially under semi-immersion conditions, on agar substrate, or both.

In some embodiments, the agar is replaced or refreshed at least once. In some embodiments, the agar is in a container or a bag. In some embodiments, the culturing comprises culturing on agar in a container or a bag.

In some embodiments, a nodal explant comprises a bud. In some embodiments, the but is obtained or derived from a mother plant. In some embodiments, the but is derived or obtained from a young mother plant. In some embodiments, the bud is obtained or derived from a cell or a tissue culture.

In some embodiments, the method further comprises a step preceding step (a) comprising obtaining or providing a nodal explant.

In some embodiments, the method further comprises a step preceding step (a) comprising obtaining or providing a bud as described herein.

In some embodiments, culturing at least partially under semi-immersion conditions, is in a semi-immersion bioreactor.

In some embodiments, the culturing is for a period of at least 3 weeks, at least 4 weeks, at least 5 weeks, at least 6 weeks, or any value and range therebetween. Each possibility represents a separate embodiment of the invention. In some embodiments, the culturing is for a period of 3 to 7 weeks.

In some embodiments, culturing under semi-immersion conditions is for a period of at least 4 days, at least 5 days, at least 6 days, at least 7 days, at least 9 days, at least 10 days, at least 12 days, at least 14 days, or any value and range therebetween. Each possibility represents a separate embodiment of the invention. In some embodiments, culturing under semi-immersion conditions is for a period of 4 to 16 days, 5 to 14 days, 7 to 15 days, or 1 to 2 weeks. Each possibility represents a separate embodiment of the invention.

In some embodiments, culturing is under controlled vegetative conditions. In some embodiments, culturing under semi-immersion conditions is under controlled vegetative conditions.

In some embodiments, culturing under semi-immersion conditions comprises is under a temperature ranging from 20° C. to 30° C., 21° C. to 28° C., 22° C. to 29° C., or 23° C. to 26° C. Each possibility represents a separate embodiment of the invention.

In some embodiments, culturing under semi-immersion conditions is under a long-day photoperiod.

In some embodiments, long-day photoperiod comprises 18 hr of light and 6 hr of dark.

In some embodiments, culturing under semi-immersion conditions results in IF development from the nodal explant.

In some embodiments, culturing under semi-immersion conditions stops or is halted once at least one IF is developed from the nodal explant.

In some embodiments, the step (b) commences once at least one IF is developed from the nodal explant.

In some embodiments, the step (b) commences if at least one IF is developed from the nodal explant.

In some embodiments, step (b) further comprises contacting the nodal explant with an effective amount of at least one elicitor selected from the group consisting of: bacterial flagella peptide (F1g22), chitin, chitosan, methyl jasmonate (MeJA), and Botrytis cinerea-derived material, and any combination thereof.

In some embodiments, step (b) further comprises contacting the nodal explant with an effective amount adenine, paclobutrazol, olivetolic acid, or any combination thereof.

In some embodiments, the method further comprises a step (c) comprising culturing the nodal explant resulting from step (b) under controlled flowering conditions.

In some embodiments, controlled flowering conditions comprises culturing under a temperature ranging from 20° C. to 30° C., 21° C. to 28° C., 22° C. to 29° C., or 23° C. to 26° C.

In some embodiments, culturing under controlled flowering conditions is under a short-day photoperiod.

In some embodiments, short-day photoperiod comprises 12 hr of light and 12 hr of dark.

In some embodiments, culturing under controlled flowering conditions is for a period of at least 21 days, at least 23 days, at least 25 days, at least 26 days, at least 27 days, at least 28 days, at least 30 days, at least 35 days, or any value and range therebetween. Each possibility represents a separate embodiment of the invention. In some embodiments, culturing under semi-immersion conditions is for a period of 2 to 5 weeks, 3 to 5 weeks, 3 to 4 weeks, or 4 to 6 weeks. Each possibility represents a separate embodiment of the invention.

In some embodiments, the method further comprises harvesting at least one IF from the tissue cultured Cannabis plantlet.

In some embodiments, the harvesting is during the culturing, after the culturing, or both. In some embodiments, the harvesting is after step (c).

As used herein, bacterial flagella peptide (F1g22) refers to a 22 amino acid long bacterial flagella peptide.

In some embodiments, chitin is a chitin obtained or derived from a fungus cell wall.

In some embodiments, Botrytis cinerea-derived material comprises an extract, a lysate, a homogenate, any fraction thereof, or any combination thereof, being derived from B. cinerea.

In some embodiments, an effective amount of Flg22 refers to a concentration of 0.1 to 10 μM, as exemplified herein.

In some embodiments, an effective amount of chitin refers to 0.1 to 10 g/L, as exemplified herein.

In some embodiments, an effective amount of chitosan refers to 0.1 to 10 g/L, as exemplified herein.

In some embodiments, an effective amount of methyl jasmonate (MeJA) refers to 0.05 to 1 mM, as exemplified herein.

In some embodiments, an effective amount of B. cinerea-derived material, such as an extract, refers to 4 mg/L extract, as exemplified herein.

In some embodiments, the method is directed to, inter alia, culturing for direct flowering. In some embodiments, the method is devoid of vegetative establishment of a plantlet as disclosed herein.

According to some embodiments, there is provided a method for controlling the production profile of at least one cannabinoid, at least one terpene, at least one flavonoid, or any combination thereof in a tissue cultured Cannabis.

In some embodiments, controlling the production profile comprises increasing the reproducibility of the production profile. In some embodiments, the method comprises culturing the tissue cultured Cannabis plantlet of the invention under conditions suitable for producing at least one cannabinoid, at least one terpene, at least one flavonoid, or any combination thereof. In some embodiments, the method comprises culturing a plurality of tissue cultured Cannabis plantlets as disclosed herein, under conditions suitable for producing at least one cannabinoid, at least one terpene, at least one flavonoid, or any combination thereof.

In some embodiments, at least 20%, at least 30%, at least 40%, at least 50%, at least 60%, at least 70%, at least 80%, at least 90%, at least 90%, or at least 99% of the IF of the tissue cultured Cannabis plantlet of the invention produce essentially the same profile of at least one cannabinoid, at least one terpene, at least one flavonoid, or any combination thereof, or any value and range therebetween. Each possibility represents a separate embodiment of the invention.

In some embodiments, at least 20%, at least 30%, at least 40%, at least 50%, at least 60%, at least 70%, at least 80%, at least 90%, at least 90%, or at least 99% of the plurality of tissue cultured Cannabis plantlets as disclosed herein produce essentially the same profile of at least one cannabinoid, at least one terpene, at least one flavonoid, or any combination thereof, or any value and range therebetween. Each possibility represents a separate embodiment of the invention.

As used herein, the term “essentially the same” refers to at least 50%, at least 60%, at least 70%, at least 80%, at least 90%, at least 95%, or at least 99% similarity, or any value and range therebetween. Each possibility represents a separate embodiment of the invention.

In some embodiments, essentially the same comprises 100% similarity.

As used herein, the terms “similarity” and “identity” are interchangeable.

In some embodiments, at least 20%, at least 30%, at least 40%, at least 50%, at least 60%, at least 70%, at least 80%, at least 90%, at least 90%, or at least 99% of the plurality of tissue cultured Cannabis plantlets as disclosed herein produce the same profile of at least one cannabinoid, at least one terpene, at least one flavonoid, or any combination thereof, or any value and range therebetween. Each possibility represents a separate embodiment of the invention.

In some embodiments, the method comprising providing and/or subjecting the tissue cultured Cannabis plantlet to conditions suitable for modifying the production profile of at least one cannabinoid, at least one terpene, at least one flavonoid, or any combination thereof, in the tissue.

In some embodiments, the method further comprises a step of producing the tissue cultured Cannabis plantlet.

In some embodiments, the conditions comprise: light, radiation, temperature, gas exchange rate, nutrients, hormones, elicitors, metabolic precursors, media, or any combination thereof.

In some embodiments, the conditions are amendable such that the production profile of at least one cannabinoid, at least one terpene, at least one flavonoid, or any combination thereof, in the tissue, is controllable.

In some embodiments, the herein disclosed method provides a highly reproducible and/or uniform production profile of at least one cannabinoid, at least one terpene, at least one flavonoid, or any combination thereof, in the tissue. In some embodiments, the tissue is an inflorescence. In some embodiments, the inflorescence is a plurality of inflorescences. In some embodiments, at least 50%, at least 60%, at least 70%, at least 80%, at least 90%, at least 95%, at least 97%, at least 99%, or 100% of inflorescences of a plurality of inflorescence in a culture as described herein, or any value and range therebetween, produce an identical or essentially the same profile of at least one cannabinoid, at least one terpene, at least one flavonoid, or any combination thereof. Each possibility represents a separate embodiment of the invention.

In some embodiments, at least 90%, at least 95%, at least 97%, at least 99%, or 100% of inflorescences of a plurality of inflorescence in a culture as described herein, or any value and range therebetween, produce an identical or essentially the same profile of at least one cannabinoid, at least one terpene, at least one flavonoid, or any combination thereof. Each possibility represents a separate embodiment of the invention.

In some embodiments, 80-100%, 85-99%, 90-97%, 92-99%, 95-100% of inflorescences of a plurality of inflorescence in a culture as described herein, produce an identical or essentially the same profile of at least one cannabinoid, at least one terpene, at least one flavonoid, or any combination thereof. Each possibility represents a separate embodiment of the invention.

In some embodiments, modifying comprises increasing or decreasing.

In some embodiments, modifying is compared to a control mother plant. In some embodiments, a control mother plant comprises a control IF of a mother plant.

In some embodiments, controlling the production comprises inducing the tissue cultured Cannabis plantlet to produce a predetermined profile of the at least one cannabinoid, said at least one terpene, said at least one flavonoid, or any combination thereof.

In some embodiments, the tissue cultured Cannabis plantlet is modifiable or controllable. In some embodiments, “modifiable” or “controllable” is in the sense that it produces a predetermined profile of at least one cannabinoid, said at least one terpene, said at least one flavonoid, or any combination thereof.

In some embodiments, the “modifiable” or “controllable” tissue cultured Cannabis plantlet produces or synthesizes at least 5% by weight more, at least 15% by weight more, at least 25% by weight more, at least 50% by weight more, at least 75% by weight more, at least 100% by weight more, at least 250% by weight more, at least 500% by weight more, at least 750% by weight more, or at least 1,000% by weight more, at least one cannabinoid, at least one terpene, at least one flavonoid, or any combination thereof, compared to a control inflorescence of a mother plant, or any value and range therebetween. Each possibility represents a separate embodiment of the invention.

In some embodiments, the “modifiable” or “controllable” tissue cultured Cannabis plantlet produces or synthesizes at most 5% by weight more, at most 15% by weight more, at most 25% by weight more, at most 50% by weight more, at most 75% by weight more, at most 100% by weight more, at most 250% by weight more, at most 500% by weight more, at most 750% by weight more, or at most 1,000% by weight more, at least one cannabinoid, at least one terpene, at least one flavonoid, or any combination thereof, compared to a control inflorescence of a mother plant, or any value and range therebetween. Each possibility represents a separate embodiment of the invention.

Methods for determining the amounts of phytochemicals, e.g., cannabinoids, terpenes, and flavonoids, are common and would be apparent to one of ordinary skill in the art. A non-limiting example for a method of quantifying such phytochemical includes, but is not limited to, gas chromatography mass spectrometry (GC-MS).

In some embodiments, the tissue cultured Cannabis plantlet is genetically modified. In some embodiments, the tissue cultured Cannabis plantlet is a genetically or genomically modified. In some embodiments, the tissue cultured Cannabis plantlet comprises a transgene. In some embodiments, the tissue cultured Cannabis plantlet is a transgenic plantlet. In some embodiments, the tissue cultured Cannabis plantlet is genetically edited.

Methods for genetic modification and/or editing are common and would be apparent to one of ordinary skill in the art. Non-limiting example for a method of genetic editing includes, but is not limited to, the use of a CRISPR-Cas system.

In some embodiments, the tissue cultured Cannabis plantlet or leaves thereof comprise at least 5% by weight less, at least 15% by weight less, at least 25% by weight less, at least 35% by weight less, at least 50% by weight less, at least 65% by weight less, at least 70% by weight less, at least 80% by weight less, at least 90% by weight less, at least 95% by weight less, or 100% by weight less, cellulose, cutin, suberin, wax, or any combination thereof, per 1 gr of the tissue cultured Cannabis plantlet compared to 1 gr of a control a mother plant or a part thereof, or any value and range therebetween. Each possibility represents a separate embodiment of the invention.

In some embodiments, a part of a control mother plant comprises a leaf. In some embodiments, a leaf comprises a sugar leaf, a fan leaf, or a combination thereof.

In some embodiments, the tissue cultured Cannabis plantlet or a leaf thereof is characterized by having a cuticle being at least 5% less thick, at least 15% less thick, at least 25% less thick, at least 35% less thick, at least 50% less thick, at least 65% less thick, at least 70% less thick, at least 80% less thick, at least 90% less thick, at least 95% less thick, or 100% less thick, compared to a cuticle of a control mother plant or part thereof, or any value and range therebetween. Each possibility represents a separate embodiment of the invention.

In some embodiments, the IF of a tissue cultured Cannabis plantlet as disclosed herein is characterized by having a stigma at least 5%, at least 15%, at least 25%, at least 50%, at least 75%, or at least 100% longer or shorter, compared to a stigma of a control inflorescence of a mother plant, or any value and range therebetween. Each possibility represents a separate embodiment of the invention.

In some embodiments, the tissue cultured Cannabis plantlet disclosed herein comprises IF being characterized by having at least 5%, at least 15%, at least 25%, at least 50%, at least 75%, or at least 100% more trichomes per 1 cm 2, compared to a control inflorescence of a mother plant, or any value and range therebetween. Each possibility represents a separate embodiment of the invention.

In some embodiments, sugar leaves of the tissue cultured Cannabis plantlet disclosed herein are characterized by having at least 5%, at least 15%, at least 25%, at least 50%, at least 75%, or at least 100% more trichomes per 1 cm 2, compared to a control sugar leaves of a mother plant, or any value and range therebetween. Each possibility represents a separate embodiment of the invention.

In some embodiments, the tissue cultured Cannabis plantlet comprises a residual amount of at least one hormone, at least one elicitor, or any combination thereof.

In some embodiments, a residual amount comprises 0.001% to 1%, 0.01% to 1%, 0.1% to 1%, 0.005% to 1%, 0.05% to 1%, 0.5% to 1%, 0.025% to 0.85%, 0.01% to 0.90%, 0.001% to 0.1%, 0.01% to 0.70%, by weight of the tissue. Each possibility represents a separate embodiment of the invention.

In some embodiments, the size and/or diameter of a flower on an IF of a tissue cultured Cannabis plantlet as disclosed herein is at least 5%, at least 15%, at least 25%, at least 50%, or at least 100% lower than the size and/or diameter of a flower on a control mother plant, or any value ad range therebetween. Each possibility represents a separate embodiment of the invention.

In some embodiments, the production cycle from a tissue to at least one cannabinoid, at least one terpene, at least one flavonoid, or any combination thereof, in the tissue culture as disclosed herein, is at least 5% shorter, at least 15% shorter, at least 25% shorter, at least 50% shorter, at least 75% shorter, at least 100% shorter, at least 150% shorter, at least 250% shorter, at least 350% shorter, at least 500% shorter, at least 750% shorter, at least 850% shorter, at least 900% shorter, or at least 1,000% shorter compared to a control mother plant, or any value and range therebetween. Each possibility represents a separate embodiment of the invention.

In some embodiments, the production cycle from a tissue to at least one cannabinoid, at least one terpene, at least one flavonoid, or any combination thereof, in the tissue culture as disclosed herein, ranges from 5 weeks to 9 weeks, 5 weeks to 8 weeks, 6 weeks to 8 weeks, or 7 weeks to 9 weeks. Each possibility represents a separate embodiment of the invention.

In some embodiments, the weight of a flower and/or the total weight of an inflorescence of the herein disclosed tissue cultured Cannabis plantlet, is at least 5%, at least 15%, at least 25%, at least 50%, or at least 100% lower than the weight of a flower and/or the total weight of an inflorescence of a mother plant, or any value and range therebetween. Each possibility represents a separate embodiment of the invention.

In some embodiments, the number of inflorescences on a tissue cultured Cannabis plantlet as disclosed herein is at least 5%, at least 15%, at least 25%, at least 50%, at least 75%, or at least 100% lower compared to the number of inflorescences on a mother plant, or any value and range therebetween. Each possibility represents a separate embodiment of the invention.

In some embodiments, the herein disclosed method utilizes a tissue cultured Cannabis plantlet characterized by being devoid of a root and suitable for controlled production of at least one cannabinoid, at least one terpene, at least one flavonoid, or any combination thereof, in a culture.

In some embodiments, culturing the tissue cultured Cannabis plantlet disclosed herein for controlled production of at least one cannabinoid, at least one terpene, at least one flavonoid, or any combination thereof, is performed on any substrate suitable for such production.

In some embodiments, the substrate is a liquid substrate.

In some embodiments, the substrate is a solid substrate.

In some embodiments, the substrate is a semi-solid substrate.

In some embodiments, the substrate comprise or is agar. In some embodiments, the substrate comprises water. In some embodiments, the substrate comprises nutrients. In some embodiments, the substrate comprises microelements.

In some embodiments, culturing is in a bioreactor.

In some embodiments, culturing is semi-immersion, full immersion, or a combination thereof.

In some embodiments, during culturing the plantlet disclosed herein is positioned in a vertical position.

In some embodiments, during culturing the plantlet disclosed herein is positioned in a horizontal position.

In some embodiments, the herein disclosed method is directed to induction of flowering in a tissue culture.

In some embodiments, the production profile of at least one cannabinoid, at least one terpene, at least one flavonoid, or any combination thereof in the tissue cultured Cannabis plantlet is at least 30% by weight, at least 40% by weight, at least 50% by weight, at least 60% by weight, at least 70% by weight, at least 80% by weight, at least 90% by weight, at least 95% by weight, at least 97% by weight, at least 99% by weight, or at least 100% by weight, identical to the production profile of at least one cannabinoid, at least one terpene, at least one flavonoid, or any combination thereof in a control inflorescence of a mother plant, or any value and range therebetween. Each possibility represents a separate embodiment of the invention.

In some embodiments, a control inflorescence of a mother plant comprises or is produced indoor, such as in a growth room, or in the field.

According to some embodiments, the method of the invention further comprise a step of extracting or purifying the at least one cannabinoid, the at least one terpene, the at least one flavonoid, or any combination thereof, from the tissue cultured Cannabis plantlet.

Methods for extracting and/or purifying phytochemicals, e.g., a cannabinoid, terpenes, flavonoids, or any combination thereof, from plant material, such as the tissue cultured Cannabis plantlet as disclosed herein, are common and would be apparent to one of ordinary skill in the art.

The Cannabis plant and parts thereof, as described herein, including tissue and subsequent generations derived therefrom, may be further exposed to mutagenesis and/or marker assisted selection, as is known to persons skilled in the art, to generate and/or select for new plants with desirable phenotypic, chemotypic and/or genotypic profiles. This can provide non-transgenic Cannabis plants that are free of exogenous nucleic acid molecule, thereby avoiding the restrictions that otherwise apply to genetically modified organisms (GMO), including plants, in some countries/regions. Typically, a progenitor plant cell, tissue, seed, or plant is exposed to mutagenesis to produce single or multiple point mutations, such as nucleotide substitutions, deletions, additions and/or codon modification.

According to some embodiments, there is provided a bioreactor configured to providing the tissue of the invention with conditions suitable for controlling the production profile of at least one cannabinoid, at least one terpene, at least one flavonoid, or any combination thereof, in the tissue of the invention.

According to some embodiments, there is provided a composition comprising at least one cannabinoid, at least one terpene, at least one flavonoid, or any combination thereof produced by the tissue of the invention.

According to some embodiments, there is provided a composition comprising at least one cannabinoid, at least one terpene, at least one flavonoid, or any combination thereof produced according to the herein disclosed method.

In some embodiments, the composition further comprises a pharmaceutical carrier.

In some embodiments, the composition is a pharmaceutical composition.

In some embodiments, the pharmaceutical composition is suitable for use in the treatment of a subject in need thereof.

As used herein, the terms “carrier”, “excipient”, or “adjuvant” refer to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N. J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

A pharmaceutical composition may take any physical form necessary for proper administration. The composition comprising an encapsulated one or more cannabinoid compounds can be administered in any suitable form, including but not limited to a liquid form, a gel form, a semi-liquid (e.g., a liquid, such as a viscous liquid, containing some solid) form, a semi-solid (a solid containing some liquid) form, or a solid form. Compositions can be provided in, for example, a tablet form, a capsule form, a liquid form, a food form a chewable form, a non-chewable form, a transbuccal form, a sublingual form, a slow-release form, a non-slow-release form, a sustained release form, or a non-sustained-release form.

A pharmaceutically-acceptable carrier suitable for the preparation of unit dosage form of a composition as described herein for peroral administration is well-known in the art.

In some embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), stabilizers (e.g. hydroxypropyl cellulose, hydroxypropylmethyl cellulose), viscosity increasing agents(e.g. carbomer, colloidal silicon dioxide, ethyl cellulose, guar gum lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), polymer coatings (e.g., poloxamers or poloxamines), and/or coating and film forming agents (e.g. ethyl cellulose, acrylates, polymethacrylates).

In some embodiments, preparation of effective amount or dose can be estimated initially from in vitro assays. In one embodiment, a dose can be formulated in animal models, and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature.

Materials and Methods

Flowering in Tissue Culture Protocol

Grow Cannabis s. plants in door and keep it in a vegetative conditions—long-day photoperiod (18/6 hours light/dark).

Sterile nodal explants by immersing them in 2% sodium hypochlorite with 0.1% (v/v) of triton for eight minutes and rinse in autoclaved deionized water three times.

Prepare 118 mm diameter round plastic filtered box with 65 ml MS medium: MS (M0256, Duchefa)—4.4 g/L; 3.2 Sucrose—20 g/L; 3.3 MES—0.5 g/L; 3.4 Plant agar—8 g/L; 3.5 pH—5.7-5.8.

Put five explants in each box.

Grow the explants under controlled vegetative conditions for two weeks—24° C.±1° C. under long-day photoperiod (18/6 h light/dark).

Subculture the explants to the same medium (MS) with elicitors, and 80 mg/L Adenine, 2 μM Paclobutrazol, and 2 μM olivetolic acid.

Transfer the cultures to controlled flowering conditions of −24° C.±1° C. under short-day photoperiod (12/12 h light/dark) for 4 weeks.

Harvest the inflorescent

Elicitation of Cannabinoids and Terpenes Production

Different compounds that can elicit the production of secondary metabolites are used during flowering period of asexual clone of Cannabis tissue culture plants. Compounds are embedded inside the growth medium. Elicitors used are: bacterial flagella peptide comprising 22 amino acids (F1g22), fungal cell wall component, chitin, a derivative of chitin chitosan, the plant hormone methyl jasmonate (MeJA), and plant pathogen Botrytis cinerea extract.

Different elicitors and combination tested: (1) Growth Medium without any elicitors (control); (2) Growth Medium supplemented with 1 μM Flg22 (Genscript, Rp19986); (3) Growth Medium supplemented with 100 mg/L Chitosan (Sigma C3646); (4) Growth Medium with 1 gr/L Chitin (Sigma, C7170); (5) Growth Medium supplemented with 0.3 mM MeJA (Sigma, 392707); (6) Growth Medium supplemented with B. cinerea extract (4 mg/L); (7) Growth Medium supplemented with 100 mg/L Chitosan and 1 gr/L Chitin; and (8) Growth Medium with 0.3 mM MeJA, 100 mg/L Chitosan and 1 gr/L Chitin.

Gross Morphology Characterization.

The inventors have compared same strain flowering Cannabis plants that were grown in growth room (GR) and were grown in tissue culture (TC).

Growth room—plants were grown in a long-day photoperiod (18/6 h light/dark) for 8 weeks for vegetative growth and transferred to a short-day photoperiod (12/12 h) inside growth chambers for the flowering stage.

Tissue culture—nodal explants were cultured on Murashige & Skoog media (MS M0256, Duchefa) under long-day photoperiod (18/6 h) for two weeks and transferred to short-day photoperiod (12/12 h) for flowering.

Plants were in flowering stage for the same time period in growth room and tissue culture for one month before experiments.

Fan leaves—the inventors compared the closest fan leaves to the inflorescence.

Sugar leaves—leaves with trichomes in the inflorescence.

Comparison of Number of Stomata on Abaxial Fan Leaves

Abaxial fan leaves mold was created using a silicone impression material (elite HD+, Zhermack). From it, the inventors took a pattern using nail polish and checked it under light microscope. From each leaf the inventors counted stomata in three areas (0.15 mm 2 each) along the main artery.

Cuticle Thickness

Sugar leaves were harvested from tissue culture and from growth room.

Fixation was performed as follow:

Four (4)% formaldehyde in PBS for 1 hour.

Three (3) washes for 5 min with PBS.

Twenty five (25)% sucrose overnight.

Latitude sections of the leaves were performed using cryostat and then scanned in scanning electron microscopy (SEM).

Cutin Monomers Analysis

Cutin monomers analysis for sugar leaves from tissue culture and growth room was performed as follow:

Tissue was extracted with chloroform and methanol (1:1, v:v) in a glass vial for 2 weeks. chloroform and methanol were replaced daily.

The exhaust extracted tissue was air dried at 30° C., flushed with N₂, and analyzed by gas chromatography (GC).

Chlorophyll Content Analysis

The inventors have compared the contents of chlorophyll a, chlorophyll b, and total carotenoids, in sugar leaves of plants from tissue culture and plants from growth room.

Briefly, leaves were weighted and incubated in 80% acetone solution for 24 hours. Thereafter, absorbance at wavelengths of 661.5 nm (chlorophyll a), 645 nm (chlorophyll b), and 470 nm (total carotenoids), were measured using spectrophotometer.

### Example 1

Developing a Protocol for High Efficiency Plant Propagation in Tissue Culture Systems

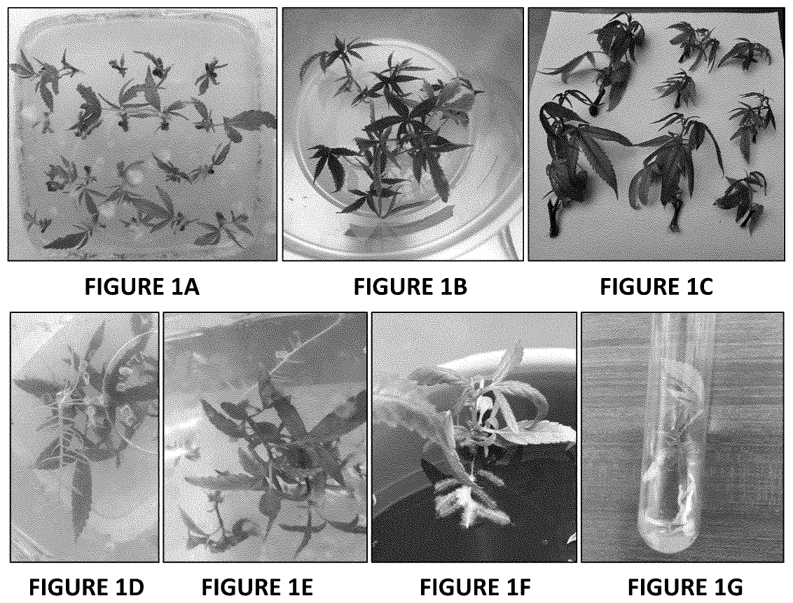

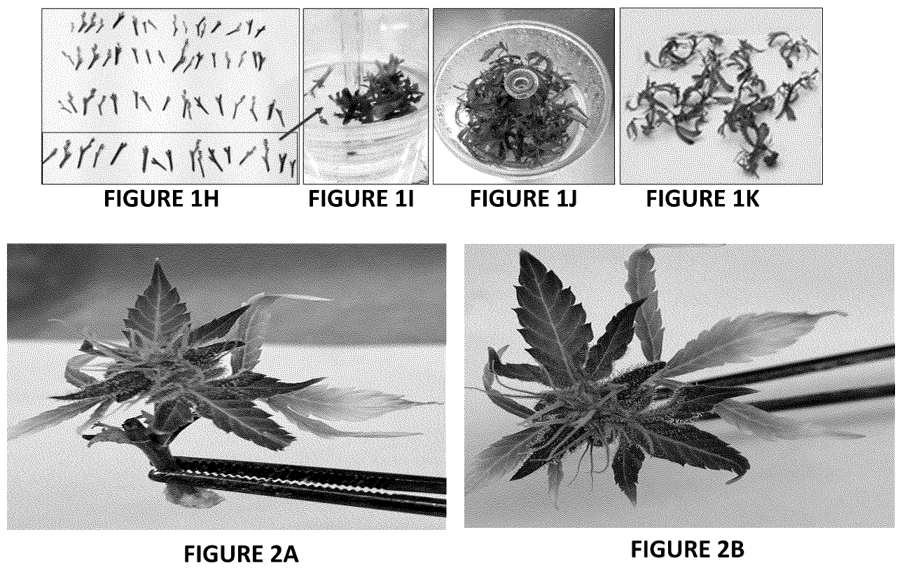

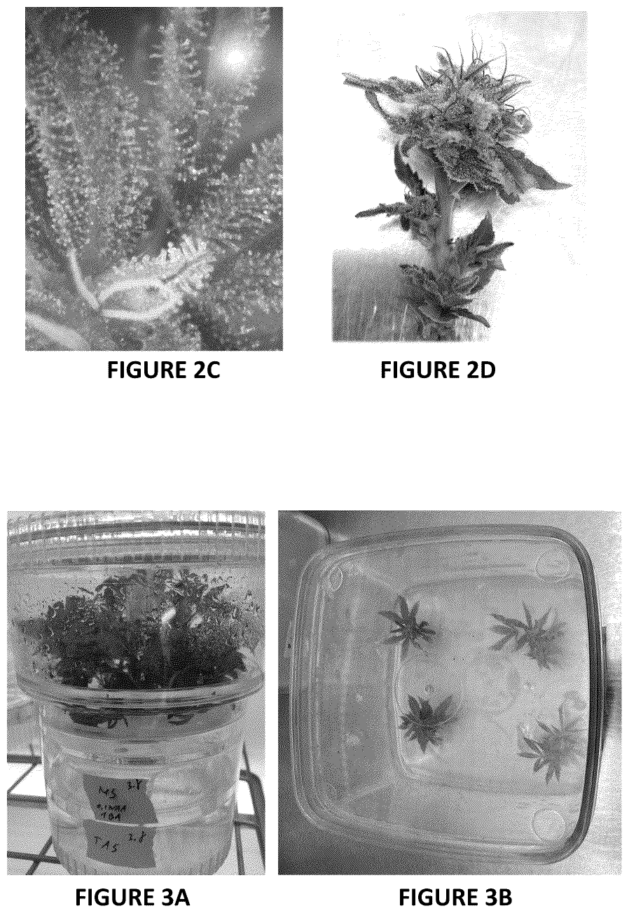

Propagation by tissue culture offer certain advantages that includes the following: only a small amount of initial plant tissue is required; new plantlets can be grown in short time and more likely to be free of diseases; only a relatively small of space is required for propagation; it is easy to transport and distribute locally and overseas. Micropropagation in Cannabis can reduce the load of maintaining mother plant population, a burden that every farm must cope with. To develop a protocol for Cannabis propagation in tissue culture the inventors took the approach of shoot multiplication, in which one node is used to propagate plantlets in several cycles of re-culturing. The inventors tested different types of culture vessels including tubes, petri dishes, magenta and conical flask. The inventors tried different filters to allow gas exchange and tested few types of media and many differ phytohormones combinations. After finding the best condition for shoot development, the inventors screened for the best condition for rooting and hardening (FIG. 1).

Another system the inventors tested for low-cost large-scale production was multiplication in a semi-immersion bioreactor (FIG. 1). Nodes in this system exhibited high rate of growth and within two weeks' plants were ready to be rooted.

### Example 2

Leaves of Tissue Cultured Plantlet Vary from Leaves of a Plant Grown in a Growth Room

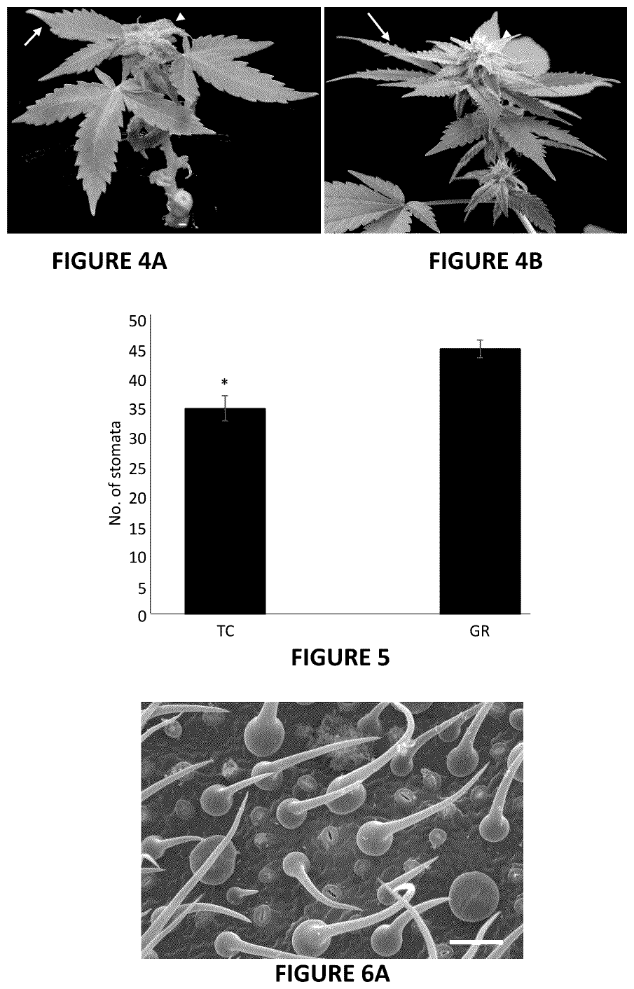

At a gross morphology analysis, the tissue cultured (TC) plantlet of the invention was shown to comprise leaves having normal development, including the production of both fan leaves and sugar leaves (FIG. 4A).

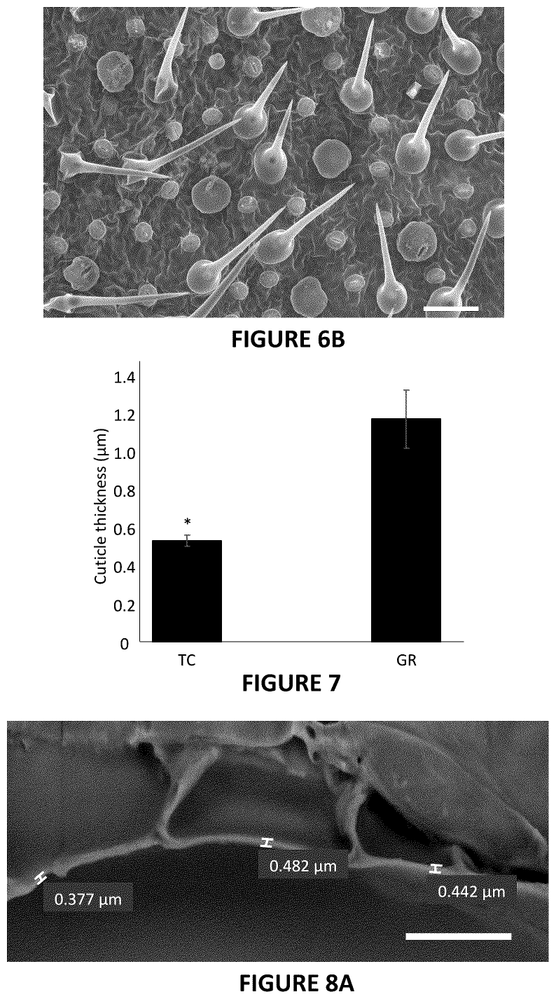

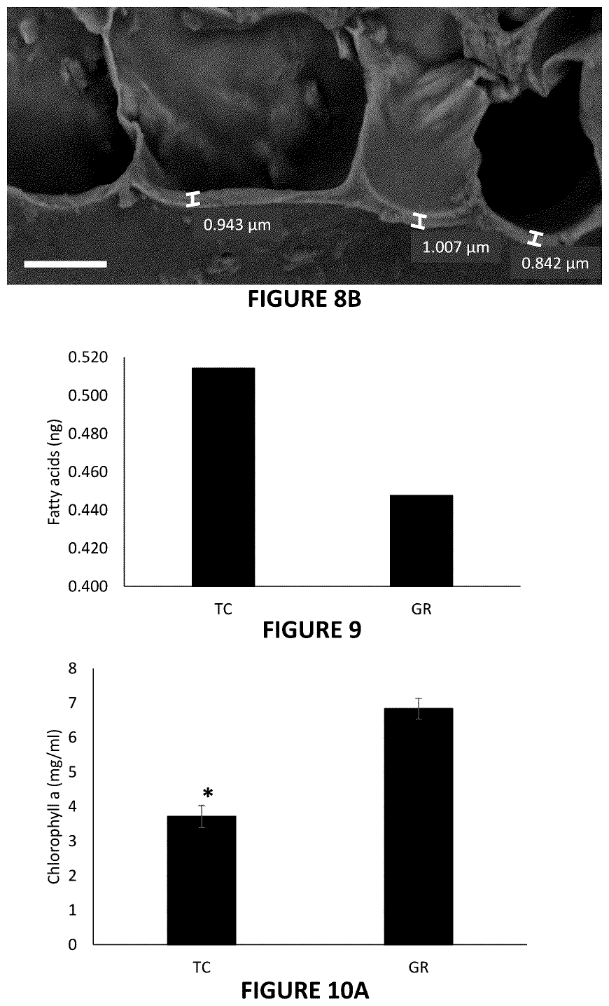

In an in depth analysis, the inventors have shown that leaves of the tissue cultured plantlet of the invention are distinguishable from these collected from control whole plants grown in a growth room. Specifically, the inventors showed that abaxial fan leaves of the TC plantlet of the invention are characterized by significantly less stomata, compared to leaves of whole plants (FIGS. 5-6). Further, the inventors showed that the cuticle of sugar leaves from the TC plantlet of the invention are significantly thinner than that of sugar leaves from control whole plants grown in a growth room (FIGS. 7-8).

Further, the inventors have examined whether the TC plantlet of the invention and the whole plants grown in a growth room are biochemically distinct.

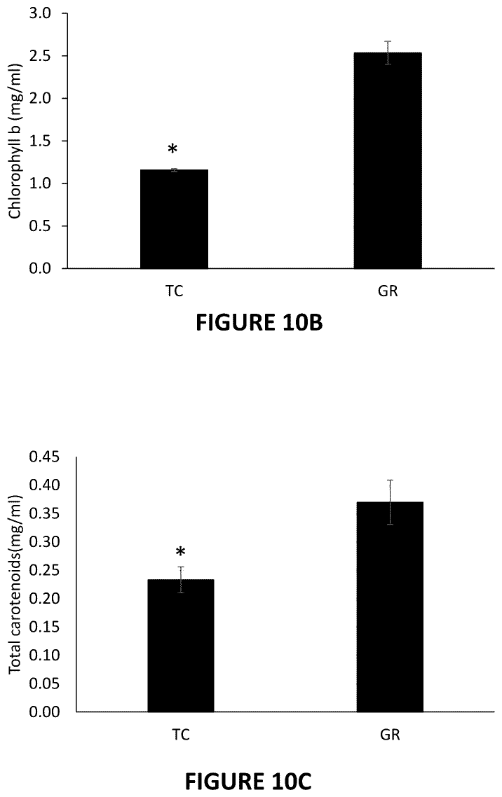

Indeed, the inventors showed that extracts of leaves derived from the TC plantlet of the invention comprise about 10% by weight more fatty acids than extracts derived from leaves derived from control whole plants grown in a growth room (FIG. 9). Also, extracts of the leaves of the TC plantlet of the invention where found to comprise 46% by weight, 55% by weight, and 37% by weight, less chlorophyll a, chlorophyll b, and total chlorophyll, respectively, compared to extracts derived from leaves derived from control whole plants grown in a growth room (FIGS. 10A-10C, respectively).

### Example 3

Physical Characterization of the Tissue Cultured Cannabis IF of the Invention

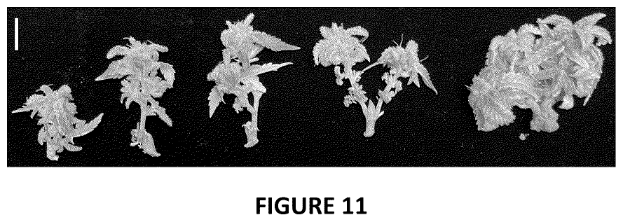

Tissue cultured Cannabis plantlets were produced as described (FIG. 11). Physical parameters, e.g., length and weight, of the tissue cultured Cannabis IF of the invention were measured and are provided in Tables 1-2 hereinbelow.

TABLE 1
Length measurements

| Stem | IF | Plantlet total | IF length/Plantlet |
|---|---|---|---|
| length (mm) | length (mm) | length (mm) | total length |
| 1 | 2 | 3 | 0.67 |
| 1.5 | 2.5 | 4 | 0.63 |
| 1 | 2.3 | 3.3 | 0.69 |
| 0.5 | 2 | 2.5 | 0.8 |
| 1.2 | 2.1 | 3.3 | 0.63 |
| 0.7 | 2.5 | 3.2 | 0.78 |
| 1.2 | 3 | 4.2 | 0.71 |
| 1 | 2 | 3 | 0.67 |
| 1 | 2.2 | 3.2 | 0.68 |
| 1.5 | 2 | 3.5 | 0.57 |
| 1 | 2 | 3 | 0.67 |
| 0.7 | 2.3 | 3 | 0.73 |

TABLE 2
Weight measurements

| Stem | IF | Plantlet total | IF weight/Plantlet |
|---|---|---|---|
| weight (gr) | weight (gr) | weight (gr) | total weight |
| 0.1 | 0.22 | 0.32 | 0.68 |
| 0.06 | 0.2 | 0.26 | 0.76 |
| 0.07 | 0.11 | 0.18 | 0.61 |
| 0.13 | 0.45 | 0.58 | 0.77 |
| 0.19 | 0.26 | 0.45 | 0.58 |
| 0.04 | 0.2 | 0.24 | 0.83 |
| 0.05 | 0.25 | 0.3 | 0.83 |
| 0.07 | 0.4 | 0.47 | 0.85 |
| 0.05 | 0.25 | 0.3 | 0.83 |
| 0.03 | 0.13 | 0.16 | 0.81 |
| 0.2 | 1.4 | 1.6 | 0.87 |
| 0.1 | 1.2 | 1.3 | 0.92 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A tissue cultured Cannabis plantlet comprising an inflorescence (IF), and characterized by any one of:
a. comprising leaves having a cuticle being at least 5% less thick compared to a cuticle of leaves of a control mother plant;
b. being devoid of a root or a rooting system; and
c. a combination of (a) and (b).

2. The tissue cultured Cannabis plantlet of claim 1, wherein said leaves of said tissue cultured Cannabis plantlet are characterized by having at least 15% by weight less pigment compared to leaves of said control mother plant.

3. The tissue cultured Cannabis plantlet of claim 2, wherein said pigment comprises: a chlorophyll a, a chlorophyll b, a carotenoid, or any combination thereof.

4. The tissue cultured Cannabis plantlet of claim 2, wherein said leaves of said tissue cultured Cannabis plantlet are characterized by having at least 35% by weight less chlorophyll a, compared to leaves of said control mother plant.

5. The tissue cultured Cannabis plantlet of claim 2, wherein said leaves of said tissue cultured Cannabis plantlet are characterized by having at least 45% by weight less chlorophyll b, compared to leaves of said control mother plant.

6. The tissue cultured Cannabis plantlet of claim 2, wherein said leaves of said tissue cultured Cannabis plantlet are characterized by having at least 25% by weight less total chlorophyll content, compared to leaves of said control mother plant.

7. The tissue cultured Cannabis plantlet of claim 1, wherein said leaves of said tissue cultured Cannabis plantlet are characterized by having at least 10% by weight more fatty acids compared to leaves of said control mother plant.

8. The tissue cultured Cannabis plantlet of claim 7, wherein the amount of said fatty acid is determined in an extract of said leaves of said tissue cultured Cannabis plantlet.

9. The tissue cultured Cannabis plantlet of claim 1, wherein said leaves comprises: fan leaves, sugar leaves, or both, of said tissue cultured Cannabis plantlet.

10. The tissue cultured Cannabis plantlet of claim 1, being devoid of a pathogen or a part derived therefrom, a pesticide, or both.

11. The tissue cultured Cannabis plantlet of claim 10, wherein said pathogen comprises: a bacterium, a fungus, a virus, a protozoan, a metazoan, or any combination thereof.

12. The tissue cultured Cannabis plantlet of claim 10, wherein said part comprises: a spore, a mycelium, an egg, a pupa, a larva, a nymph, or any combination thereof, of the pathogen.

13. The tissue cultured Cannabis plantlet of claim 1, wherein said control mother plant comprises a whole plant grown indoor or in the field.

14. A plurality of IF derived or obtained from the tissue cultured Cannabis plantlet of claim 1, characterized by a production profile of at least one cannabinoid, at least one terpene, at least one flavonoid, or any combination thereof, being essentially the same among said plurality of IF.

15. The plurality of IF of claim 14, being obtained or derived from a plurality of tissue cultured Cannabis plantlets, wherein each tissue cultured Cannabis plantlet of said plurality of tissue cultured Cannabis plantlets is characterized by any one of:
a. comprising leaves having a cuticle being at least 5% less thick compared to a cuticle of leaves of a control mother plant;
b. being devoid of a root or a rooting system; and
a combination of (a) and (b).

16. A method for producing a tissue cultured Cannabis plantlet of claim 1, the method comprising the steps of:
a. providing a nodal explant derived or obtained from a Cannabis mother plant; and
b. culturing said nodal explant under at least partially under semi-immersion conditions, on agar, or both,
thereby, producing the tissue cultured Cannabis plantlet.

17. The method of claim 16, wherein said culturing at least partially under semi-immersion conditions, is in a semi-immersion bioreactor, on agar substrate, or both.

18. The method of claim 16, wherein said step (b) further comprises contacting said nodal explant with an effective amount of at least one elicitor selected from the group consisting of: bacterial flagella peptide (F1g22), chitin, chitosan, methyl jasmonate (MeJA), and Botrytis cinerea-derived material, and any combination thereof.

19. The method of claim 16, wherein said culturing under semi-immersion condition is at least 7 days.

20. A method for controlling the production profile of at least one cannabinoid, at least one terpene, at least one flavonoid, or any combination thereof, the method comprising providing the tissue cultured Cannabis plantlet of claim 1, and subjecting said cultured Cannabis plantlet to conditions suitable for modifying the production profile of at least one cannabinoid, at least one terpene, at least one flavonoid, or any combination thereof, and optionally wherein said controlling comprises increasing the reproducibility of said produced profile of at least one cannabinoid, at least one terpene, at least one flavonoid, or any combination thereof, compared to a control mother plant.

21. (canceled)
